(19) United States
(12) Patent Application Publication
(10) Pub. No.: US 2018/0119319 A1
(43) Pub. Date: May 3, 2018

(54) FABRIC AND MANUFACTURING METHOD THEREOF

(71) Applicant: CAL-COMP BIG DATA, INC., New Taipei City (TW)

(72) Inventor: Chun-An Hsu, New Taipei City (TW)

(73) Assignee: CAL-COMP BIG DATA, INC., New Taipei City (TW)

(21) Appl. No.: 15/364,230

(22) Filed: Nov. 29, 2016

(30) Foreign Application Priority Data
Oct. 27, 2016 (CN) 201610951037.1

(51) Int. Cl. D03D 1/00; D03D 25/00; A61B 5/053

(52) U.S. Cl. CPC D03D 1/0088; D10B 2401/16; A61B 5/0537; D03D 25/005

(57) ABSTRACT

A fabric and a manufacturing method thereof are provided, where the fabric is adapted to a physiological signal detection device. The fabric includes a fabric base layer, a plurality of first yarns and a plurality of second yarns. The first yarns and the second yarns are respectively woven on the fabric base layer to present a fluffy state. The first yarns are electrically conductive. A height of each of the first yarns relative to the fabric base layer is greater than a height of each of the second yarns relative to the fabric base layer, such that the first yarns are electrically conductive between a user and the physiological signal detection device. According to the fabric and the manufacturing method thereof, the fabric is wrapped on the physiological signal detection device to provide a protection effect, which still maintains a smooth detection process of the physiological signal detection device.

### CROSS-REFERENCE TO RELATED APPLICATION

This application claims the priority benefit of China application serial no. 201610951037.1, filed on Oct. 27, 2016. The entirety of the above-mentioned patent application is hereby incorporated by reference herein and made a part of this specification.

### BACKGROUND

### Technical Field

The disclosure relates to a fabric and a manufacturing method thereof.

### Description of Related Art

A present body fat meter measures body fat in a sensing manner by using a conductive sensing piece, and a principle thereof is that the sensing piece senses a variation of a received signal during a process that a human body conducts a current, so as to calculate a body fat content, and since muscles, bones and other tissues of human body contain water, it is easy to conduct the current, and since the body fat does not contain water, current conduction is blocked. Therefore, when the current is blocked too much, it represents that the body fat content is too high, and the user's body may be too fat, and need to control the diet and weight in order to improve health.

However, after a long time usage, it is easy to deposit dirt on the conductive sensing piece, or a detection result is affected by human perspiration or water after bath. Therefore, how to provide enough protection accessories to the conductive sensing piece without affecting the detection process is an important technical issue to be considered by related technicians.

### SUMMARY

The disclosure is directed to a fabric, which is on a physiological signal detection device to provide a protection effect, and meanwhile maintain a smooth detection process of the physiological signal detection device.

The disclosure provides a fabric, which is adapted to a physiological signal detection device. The fabric includes a fabric base layer, a plurality of first yarns and a plurality of second yarns. The first yarns and the second yarns are respectively woven on the fabric base layer to present a fluffy state. The first yarns are electrically conductive. A height of each of the first yarns relative to the fabric base layer is greater than a height of each of the second yarns relative to the fabric base layer. The first yarns are electrically conductive between a user and the physiological signal detection device.

In an embodiment of the disclosure, the first yarns are subjected to a yarn steaming processing before being woven to the fabric base layer, and the second yarns are not subjected to the yarn steaming processing before being woven to the fabric base layer.

In an embodiment of the disclosure, a temperature required by the yarn steaming processing is 130 degrees Celsius.

In an embodiment of the disclosure, the first yarns and the second yarns are knitted on the fabric base layer.

In an embodiment of the disclosure, the second yarns and the fabric base layer are electrically insulated.

In an embodiment of the disclosure, the fabric base layer is formed by knitting a plurality of the second yarns.

In an embodiment of the disclosure, the first yarns and the second yarns are respectively woven on the fabric base layer to form a plurality of first regions and a plurality of second regions. The first regions are formed by the first yarns, and the second regions are formed by the second yarns. The first regions and the second regions are arranged in an array and interleaved with each other.

In an embodiment of the disclosure, the physiological signal detection device includes a main body and a plurality of conduction detecting units disposed on the main body. The fabric is sleeved on the main body, and at least a part of the first regions correspondingly contact the conduction detecting units.

The disclosure provides a method for manufacturing fabric, which includes: providing a fabric base layer; performing a yarn steaming processing to a plurality of first yarns without performing the yarn steaming processing to a plurality of second yarns, where each of the first yarns is electrically conductive, and each of the second yarns is electrically insulative; weaving the first yarns and the second yarns on the fabric base layer; and performing a dyeing and finishing processing to the fabric base layer and the first yarns and the second yarns thereon to make a height of each of the first yarns relative to the fabric base layer to be greater than a height of each of the second yarns relative to the fabric base layer.

In an embodiment of the disclosure, before the dyeing and finishing processing is performed, the first yarns and the second yarns woven on the fabric base layer have a same height.

In an embodiment of the disclosure, the first yarns are woven on the fabric base layer to form a plurality of first lugs, and the second yarns are woven on the fabric base layer to form a plurality of second lugs. The method further includes: cutting the first lugs and the second lugs such that the first yarns and the second yarns present a fluffy state.

In an embodiment of the disclosure, the method further includes knitting the second yarns to form the fabric base layer.

In an embodiment of the disclosure, the first yarns and the second yarns are knitted on the fabric base layer.

According to the above description, in the embodiment of the disclosure, the fabric is adapted to the physiological signal detection device to provide a protection function, and since in the first yarns and the second yarns woven on the fabric base layer, the first yarns are electrically conductive and the second yarns are electrically insulative, and the height of the first yarns relative to the fabric base layer is greater than the height of the second yarns relative to the fabric base layer, electrical conduction between the user and the physiological signal detection device can be implemented through the first yarns, such that the physiological signal detection device can still detect physiological signals of the user under the state of being covered by the fabric.

In order to make the aforementioned and other features and advantages of the invention comprehensible, several exemplary embodiments accompanied with figures are described in detail below.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 and FIG. 2 are respectively schematic diagrams of a fabric and a physiological signal detection device according to an embodiment of the disclosure.

FIG. 3 is a partial enlarged view of the fabric of FIG. 1.

FIG. 4 and FIG. 5 are schematic diagrams illustrating statuses of the fabric in a manufacturing process.

FIG. 6 is a flowchart illustrating a method for manufacturing fabric of the disclosure.

### DESCRIPTION OF EMBODIMENTS

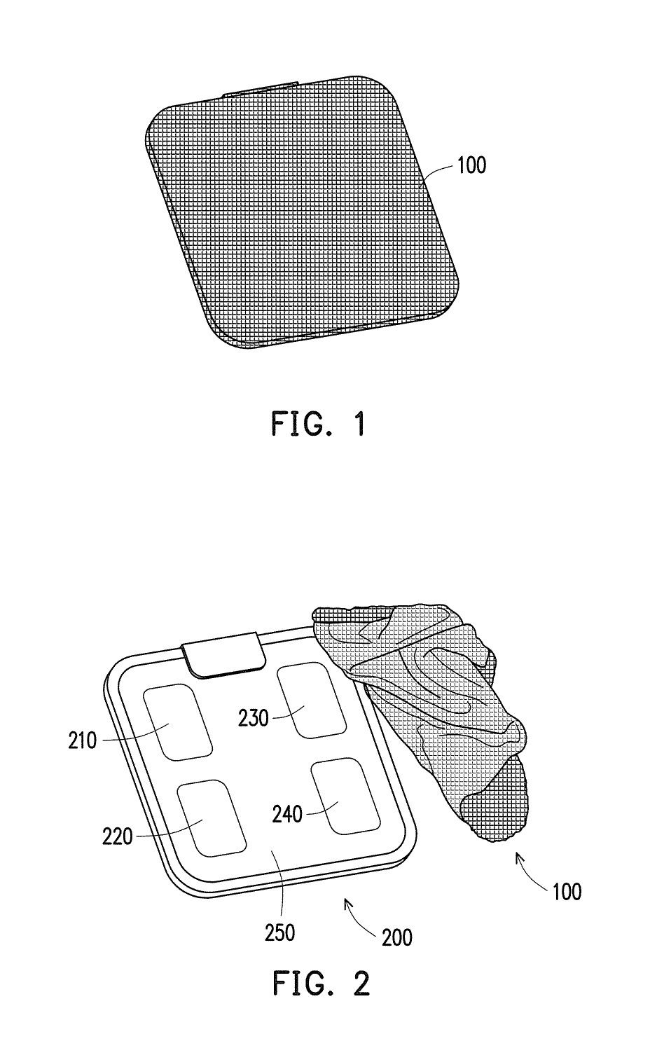

FIG. 1 and FIG. 2 are respectively schematic diagrams of a fabric and a physiological signal detection device according to an embodiment of the disclosure, where FIG. 1 is a schematic diagram of the fabric sleeving and wrapping the physiological signal detection device, and FIG. 2 is a schematic diagram of fabric lifting from the physiological signal detection device. Referring to FIG. 1 and FIG. 2, in the present embodiment, the physiological signal detection device 200 is, for example, body fat meter, which includes a main body 250 and a plurality of conduction detecting units 210-240 disposed on a surface of the main body 250. The user steps on the conduction detecting units (a single foot steps on the two conduction detecting units 230, 240 or the two conduction detecting units 210, 220), so as to measure a body fat content of the user through a process of conducting current in internal of human body. Since the structure and operations of the physiological signal detection device 200 can be learned from existing technique, detail thereof is omitted and not repeated therein.

As described above, in order to avoid influencing a detecting effect of the conduction detecting units 210-240 due to long time usage, user's status or environment, the fabric 100 of the present embodiment sleeves and wraps the main body 250 of the physiological signal detection device 200 to provide enough protection effect. Meanwhile, even the physiological signal detection device 200 is wrapped by the fabric 100, a detection operation thereof is not affected, and the fabric 100 of the present embodiment has a structure characteristic that is enough to achieve the detection operation, which is described below.

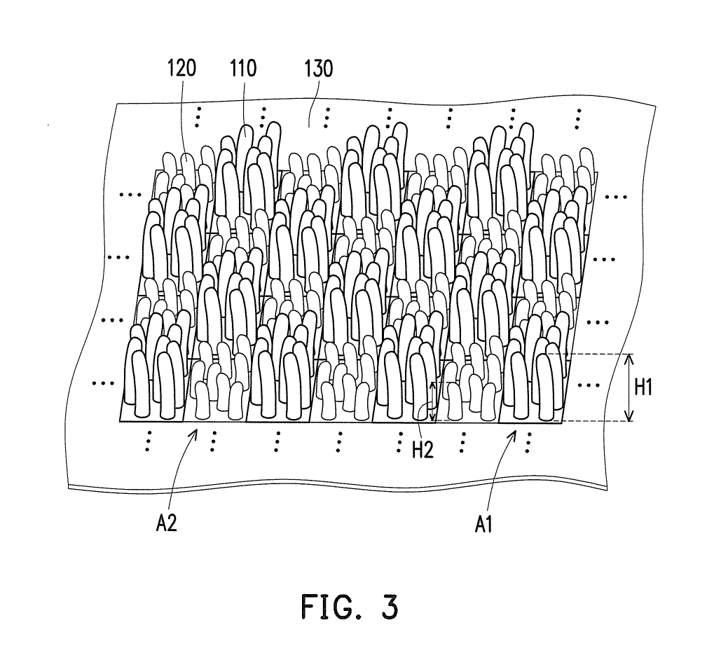

FIG. 3 is a partial enlarged view of the fabric of FIG. 1. Referring to FIG. 3, in the present embodiment, the fabric 100 includes a fabric base layer 130, a plurality of first yarns 110 and a plurality of second yarns 120. The first yarns 110 and the second yarns 120 are respectively woven on the fabric base layer 130 to form a plurality of first regions A1 and a plurality of second regions A2, where the first regions A1 are formed by the first yarns 110, and the second regions A2 are formed by the second yarns 120, and the first regions A1 and the second regions A2 are arranged in an array and are interleaved to each other.

It should be noted that the first yarns 110 are electrically conductive, and the second yarns 120 are electrically insulative, and a height H1 of each of the first yarns 110 relative to the fabric base layer 130 is greater than a height H2 of each of the second yarns 120 relative to the fabric base layer 130. In this way, when the fabric 100 covers the physiological signal detection device 200 as that shown in FIG. 1, at least a part of the first regions A1 correspondingly covers the conduction detecting units 210-240, and when the user steps thereon, the conduction detecting units 210-240 may transmit currents to the user through a plurality of the first regions A1 formed by the first yarns 110 to achieve a detection effect.

Since the height H1 of the first yarns 110 is greater than the height H2 of the second yarns 120, the first yarns 110 may first contact a sole of the user, so as to avoid interference of the contact between the human body and the first yarns 110 probably caused by the second yarns 120. On the other hand, if the heights of the first yarns and the second yarns are the same, it represents that when the user steps on the fabric, the first yarns and the human body may probably have an attenuated or weakened conductive effect there between due to the block of the second yarns.

Moreover, the first yarns 110 and the second yarns 120 of the present embodiment are knitted on the fabric base layer 130, and the fabric base layer 130 is, for example, formed by knitting a plurality of second yarns 120 to achieve the electrical insulation property.

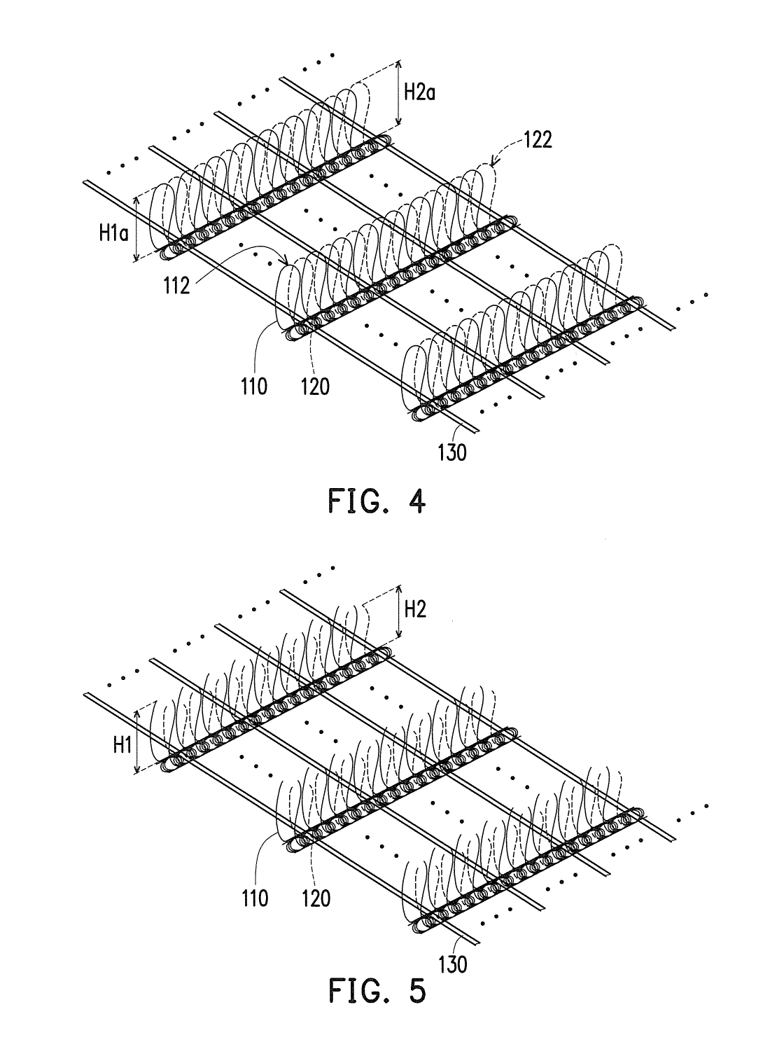

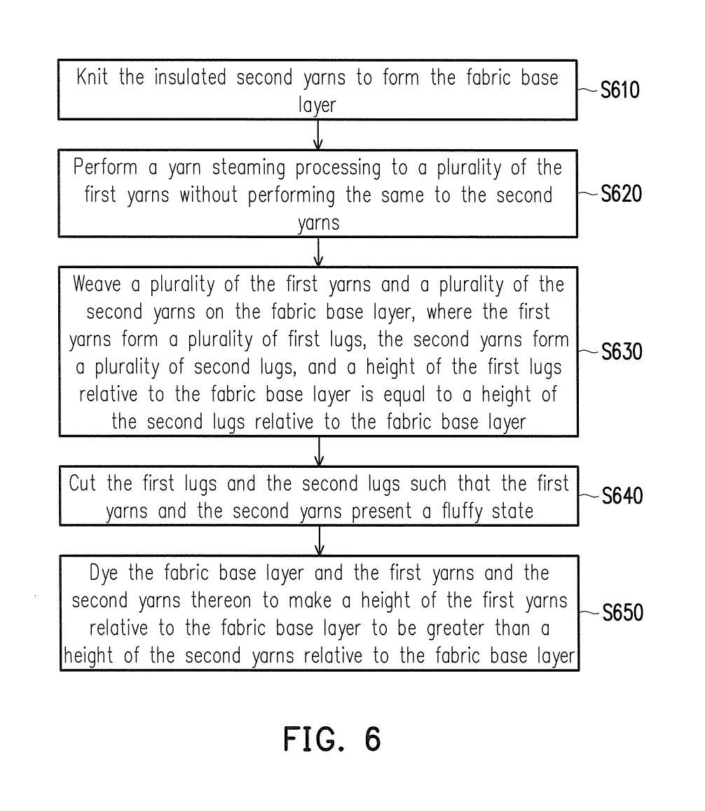

FIG. 4 and FIG. 5 are schematic diagrams illustrating statuses of the fabric in a manufacturing process. FIG. 6 is a flowchart illustrating a method for manufacturing fabric of the invention. It should be noted that only a part of the fabric 100 is shown in FIG. 4 and FIG. 5, where the fabric base layer 130 only shows a partial yarn structure to facilitate recognition. Referring to FIG. 4 to FIG. 6, in the present embodiment, in step S610, the insulated second yarns 120 are first knitted to form the fabric base layer 130. Then, in step S620, a yarn steaming processing is performed to a plurality of the first yarns 110 without performing the same to the second yarns 120. In this way, after the yarn steaming processing (after a high temperature), the first yarns 110 may have a shrinkage phenomenon, and after the shrinkage of the first yarns 110, the first yarns 110 are not further shrunk even if they are again in a high temperature state. A yarn steaming temperature is about 130 degrees Celsius.

Then, in the step S630, a plurality of the first yarns 110 and a plurality of the second yarns 120 are woven on the fabric base layer 130 to form a plurality of first lugs 112 (formed by the first yarns 110) and a plurality of second lugs (formed by the second yarns 120) on the fabric base layer 130, as shown in FIG. 4. In the step S630, controlled by means of knitting, a height H1a of the first lugs 112 relative to the fabric base layer 130 is equal to a height H2a of the second lugs 122 relative to the fabric base layer 130.

Then, in step S640, the first lugs 112 and the second lugs 122 are cut such that the first yarns 110 and the second yarns 120 present a fluffy state on the fabric base layer 130. Finally, in step S650, a dyeing and finishing processing is performed to the fabric base layer 130 and the first yarns 110, the second yarns 120 thereon, and now under the high temperature, the second yarns 120 are shrunk, though as described above, the first yarns 110 processed with the yarn steaming processing are not further shrunk in the step S650. In this way, the height H1 of the first yarns 110 relative to the fabric base layer 130 is greater than the height H2 of the second yarns 120 relative to the fabric base layer 130, as shown in FIG. 5.

In summary, in the embodiments of the disclosure, the fabric is adapted to sleeve on the main body of the physiological signal detection device to provide a protection function. In order to ensure a normal operation of the physiological signal detection device sleeved by the fabric, in the fabric base layer and the first yarns, the second yarns woven thereon, the first yarns are electrically conductive and the second yarns are electrically insulative, and the height of the first yarns relative to the fabric base layer is greater than the height of the second yarns relative to the fabric base layer, such that when the user steps on the fabric, the shorter second yarns do not influence the conductivity between the human body and the first yarns, and the conduction detecting units of the physiological signal detection device may transmit currents to the human body through the first yarns to implement the detection.

In the method for manufacturing fabric, by performing the yarn steaming processing to the first yarns before weaving, and not to perform the same to the second yarns, the first yarns may first shrink. Then, the heights of the first yarns and the second yarns are controlled to be equivalent during weaving, and finally the dyeing and finishing processing is performed to the fabric to shrink the second yarns that are not processed with the yarn steaming processing, where the first yarns are not shrunk. In this way, on the final fabric base layer, the height of the first yarns is greater than the height of the second yarns, so as to achieve the aforementioned structure characteristic.

It will be apparent to those skilled in the art that various modifications and variations can be made to the structure of the invention without departing from the scope or spirit of the invention. In view of the foregoing, it is intended that the invention cover modifications and variations of this invention provided they fall within the scope of the following claims and their equivalents.

What is claimed is:

1. A fabric, adapted to a physiological signal detection device, the fabric comprising:
a fabric base layer; and
a plurality of first yarns and a plurality of second yarns, respectively woven on the fabric base layer to present a fluffy state, wherein the first yarns are electrically conductive, and a height of each of the first yarns relative to the fabric base layer is greater than a height of each of the second yarns relative to the fabric base layer, such that the first yarns are electrically conductive between a user and the physiological signal detection device.

2. The fabric as claimed in claim 1, wherein the first yarns are subjected to a yarn steaming processing before being woven to the fabric base layer, and the second yarns are not subjected to the yarn steaming processing before being woven to the fabric base layer.

3. The fabric as claimed in claim 2, wherein a temperature required by the yarn steaming processing is 130 degrees Celsius.

4. The fabric as claimed in claim 1, wherein the first yarns and the second yarns are knitted on the fabric base layer.

5. The fabric as claimed in claim 1, wherein the second yarns and the fabric base layer are electrically insulated.

6. The fabric as claimed in claim 1, wherein the fabric base layer is formed by knitting a plurality of the second yarns.

7. The fabric as claimed in claim 1, wherein the first yarns and the second yarns are respectively woven on the fabric base layer to form a plurality of first regions and a plurality of second regions, the first regions are formed by the first yarns, and the second regions are formed by the second yarns, and the first regions and the second regions are arranged in an array and interleaved with each other.

8. The fabric as claimed in claim 7, wherein the physiological signal detection device comprises a main body and a plurality of conduction detecting units disposed on the main body, the fabric is sleeved on the main body, and at least a part of the first regions correspondingly contact the conduction detecting units.

9. A method for manufacturing fabric, comprising:
providing a fabric base layer;
performing a yarn steaming processing to a plurality of first yarns without performing the yarn steaming processing to a plurality of second yarns, wherein each of the first yarns is electrically conductive, and each of the second yarns is electrically insulative;
weaving the first yarns and the second yarns on the fabric base layer; and
performing a dyeing and finishing processing to the fabric base layer and the first yarns and the second yarns thereon to make a height of each of the first yarns relative to the fabric base layer to be greater than a height of each of the second yarns relative to the fabric base layer.

10. The method for manufacturing fabric as claimed in claim 9, wherein before the dyeing and finishing processing is performed, the first yarns and the second yarns woven on the fabric base layer have a same height.

11. The method for manufacturing fabric as claimed in claim 9, wherein the first yarns are woven on the fabric base layer to form a plurality of first lugs, and the second yarns are woven on the fabric base layer to form a plurality of second lugs, and the method for manufacturing fabric further comprises:
cutting the first lugs and the second lugs such that the first yarns and the second yarns present a fluffy state.

12. The method for manufacturing fabric as claimed in claim 9, further comprising:
knitting the second yarns to form the fabric base layer.

13. The method for manufacturing fabric as claimed in claim 9, wherein the first yarns and the second yarns are knitted on the fabric base layer.
